(19) United States
(12) Patent Application Publication
(10) Pub. No.: US 2018/0117080 A1
(43) Pub. Date: May 3, 2018

(54) COMPOSITIONS AND METHODS FOR TREATING SKIN CONDITIONS

(71) Applicants: Avi Shani, Kfar Haoranim (IL); Adva Baron, Ezer (IL); Amir Shapira, Herzeliya (IL)

(72) Inventors: Avi Shani, Kfar Haoranim (IL); Adva Baron, Ezer (IL); Amir Shapira, Herzeliya (IL)

(21) Appl. No.: 15/566,027

(22) PCT Filed: Apr. 13, 2016
(86) PCT No.: PCT/IB2016/000560

§ 371 Date: Oct. 12, 2017

Related U.S. Application Data
(60) Provisional application No. 62/146,770, filed on Apr. 13, 2015.

(51) Int. Cl. A61K 33/14; A61K 9/107; A61K 33/06; A61K 9/00; A61K 9/70; A61P 17/00; A61P 17/10

(52) U.S. Cl. CPC A61K 33/14; A61K 9/107; A61K 33/06; A61P 17/10; A61K 9/7015; A61P 17/00; A61K 9/0014

(57) ABSTRACT

The present invention provides a composition comprising: a. a water-based polymer emulsion from 10 wt % to 90 wt % of the composition; and b. Dead Sea salt from 5 wt % to 80 wt % of the composition.

### CROSS-REFERENCE TO RELATED APPLICATIONS

This application claims priority to U.S. Provisional Application Ser. No. 62/146,770, filed on Apr. 13, 2015, the entire contents of which is incorporated by reference in its entirety.

### FIELD OF THE INVENTION

The present invention relates to compositions and methods of use thereof for treating skin disorders.

### SUMMARY

In one embodiment, the present invention provides a composition comprising
a. a water-based polymer emulsion from 10 wt % to 90 wt % of the composition; and
b. Dead Sea salt from 5 wt % to 80 wt % of the composition.

In one embodiment, the composition further comprises a wetting and dispersing additive up to 20 wt % of the composition. In one embodiment, the wetting and dispersing additive is a wetting and dispersing additive for water-based coatings. In one embodiment, the wetting and dispersing additive is a wetting and dispersing additive for printing inks, coating, paints, adhesives, sealants. In one embodiment, the wetting and dispersing additive is a wetting and dispersing additive for cosmetic preparations. In one embodiment, the wetting and dispersing additive is the wetting and dispersing additive sold under the tradename DISPERBYK®.

In one embodiment, the composition further comprises a smectite clay up to 30 wt % of the composition. In one embodiment, the smectite clay is the smectite clay sold under the tradename BENTONE® EW.

In one embodiment, the composition further comprises a rheology modifier up to 30 wt % of the composition. In one embodiment, the rheology modifier is the non-ionic thickener based on polyurethane sold under the tradename TEGO® Rheo8600.

In one embodiment, the composition further comprises a defoamer up to 8 wt % of the composition. In one embodiment, the defoamer is the defoamer sold under the tradename TEGO® Foamex825.

In one embodiment, the composition further comprises water up to 90 wt % of the composition.

In one embodiment, the composition further comprises at least one additional component selected from the group consisting of: a humectant, an alcohol, an adhesion promoter, a lubricant, a softening agent, a fragrance, and a therapeutic agent.

In one embodiment, the pigment is up to 20 wt % of the composition.

In one embodiment, the fragrance is up to 5 wt % of the composition.

In one embodiment, the therapeutic agent is an anti-bacterial agent. In one embodiment, the antibacterial agent is salicylic acid. In one embodiment, the salicylic acid is up to 10 wt % of the composition.

In one embodiment, the antibacterial agent is titanium dioxide. In one embodiment, the titanium dioxide is up to 15 wt % of the composition.

In one embodiment, the present invention is a method, comprising:
a. applying the composition to a surface of the skin of a patient suffering from a skin condition, at a site in need of treatment thereof; and
b. allowing the composition to form a solid film at the site where the composition is applied;
   wherein the solid film comprises a concentration of Dead Sea salts at a concentration that is greater than the concentration of the Dead Sea salts in the composition, when the composition was first applied to the skin.

In one embodiment, the Dead Sea salts in the film permeates the skin of the patient.

In one embodiment, the permeation of the Dead Sea salts treats the skin condition.

In one embodiment, the composition is left on the skin for a time sufficient to treat the skin condition.

In one embodiment, the composition is applied in an amount effective to treat the skin condition.

In one embodiment, the skin condition is selected from the group consisting of: acne rosacea, psoriasis, rubor, tumor, calor, dolor, scarring, dry skin, aging, wrinkles, inflammation, bacerial infection, and viral infection.

In one embodiment, the present invention is a method, comprising:
a. applying the composition to a surface of the skin of a patient suffering from comedones, at a site in need of treatment thereof
   wherein the comedones are attached to the skin of the patient;
b. allowing the composition to form a solid film at the site where the composition is applied,
   wherein the solid film comprises a concentration of Dead Sea salts at a concentration that is greater than the concentration of the Dead Sea salts in the composition, when the composition was first applied to the skin,
   wherein the solid film adheres to the skin and to the comedones;
c. leaving the solid film in place for a time sufficient to detach the comedones from the skin of the patient; and
d. removing the solid film, thereby removing the comedones attached to the solid film.

In one embodiment, the time sufficient to detach the comedones from the skin of the patient is from 1 to 3 hours.

In one embodiment, the method is repeated at least once.

### BRIEF DESCRIPTION OF THE DRAWINGS

FIG. 1 shows a representation of the structure of smectite clay in a composition according to some embodiments of the present invention.

FIG. 2 shows a depiction of the mode of action of a composition according to some embodiments of the present invention.

FIG. 3 shows the diffusion of mineral salts in to the skin according to some embodiments of the present invention.

FIG. 4 shows the skin of a patient before and after treatment according to some embodiments of the present invention.

FIG. 5 shows the skin of a patient before and after treatment according to some embodiments of the present invention.

FIG. 6 shows the skin of a patient before and after treatment according to some embodiments of the present invention.

FIG. 7 shows the skin of a patient before and after treatment according to some embodiments of the present invention.

FIG. 8 shows the skin of a patient before and after treatment according to some embodiments of the present invention.

FIG. 9 shows the skin of a patient before and after treatment according to some embodiments of the present invention.

FIG. 10 shows the skin of a patient before and after treatment according to some embodiments of the present invention.

FIG. 11 shows the relationship between absolute resistivity and salt concentration in a composition according to some embodiments of the present invention.

### DETAILED DESCRIPTION

For clarity of disclosure, and not by way of limitation, the detailed description of the invention is divided into the following subsections that describe or illustrate certain features, embodiments or applications of the present invention.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

### Compositions According to Some Embodiments of the Present Invention

In some embodiments, the composition is a topical composition and provides local, continuous, and prolonged delivery of therapeutic solutes for the treatment of skin conditions. In some embodiments, the therapeutic solutes are provided by Dead Sea salts. As used herein, the term “Dead Sea salt” refers to mineral salts extracted from the Dead Sea.

In some embodiments, the present invention provides a composition comprising
a. a water-based polymer emulsion from 10 wt % to 90 wt % of the composition; and
b. Dead Sea salt from 5 wt % to 80 wt % of the composition.

Mineral Salts: In some embodiments, the mineral salts comprise Dead Sea salts. In some embodiments, the Dead Sea salt is extracted from mud obtained from the shores of the Dead Sea. In some embodiments, the mud comprises minerals (expressed in the equivalent oxides that do not occur in free form in the mud): 20% silicon dioxide, 15.5% calcium oxide, 4.8% aluminum oxide, 4.5% magnesium oxide, 2.8% iron (III) oxide, 1.7% sodium oxide, 1.3% potassium oxide, 0.5% titanium (IV) oxide, 0.4% titanium oxide, 0.4% sulphur trioxide, 0.3% phosphorous pentoxide, 6.6% chloride, and 0.2% bromide.

The salt concentration in the water of the Dead Sea is about 34% salt (variable, depending on the season) that is 8 times relative to sea water salt concentration. The overall concentration of Dead Sea salt in all the different active formulations of the present invention is in the range of 47 -52.9% w/w.

When calculating the Dead Sea salt concentration only in the formulation liquid, which is mainly water, it is in the range 70-79.5% w/w, that Is above 2 times then the Dead Sea Salt total concentration in the dead sea water. Also this concentration is well above the maximum water solubility of the different minerals in the salts as follows:

| | | | g in 100 ml | | g/L Water | formula | gr/L in |
|---|---|---|---|---|---|---|---|
| | | Results % | Dead Sea | g/L in | Solubility | content | formula |
| | | August 2012 | water | Dead Sea | 20c | gr | liquids |
| Mineral | Spec. % | * | ***** | water | ** | *** | **** |
| MgCl2 | 31.0-35.0 | 34.09 | 11.6 | 116 | 543 | 16.8 | 559.4 |
| KCl | 24.0-26.0 | 24.78 | 8.43 | 84.3 | 344 | 12.2 | 407.1 |
| NaCl | 4.0-8.0 | 4.18 | 1.42 | 14.2 | 359 | 2.06 | 68.6 |
| CaCl2 | 0.4-0.6 | 0.4 | 0.136 | 1.36 | 908 | 0.197 | 6.56 |
| Bromide | 0.3-0.6 | 0.32 | — | — | — | — | — |
| Sulphate | 0.05-0.2 | 0.09 | 0.03 | 0.3 | 139 | 0.044 | 1.47 |
| Water of | 34.0-38.0 | 34.4 | 11.7 | 117 | — | 16.95 | 564 |
| Crystall. | | | | | | | |
| Insolubles | 0.05-0.3 | 0.07 | — | — | — | — | — |

* Chemisar Laboratories INC. Guelph, Ontario, Canada, Report No. C32461, Sep. 14, 2012
** Wikipedia
*** calculated content in 100 gr formulation SC-50-15, calculated according to the above results*.
**** Calculated content in the formulation SC-50-15 liquids only (30 ml).
***** calculation based on 34% salt concentration in Dead Sea water.
* Chemisar Laboratories INC. Guelph, Ontario, Canada, Report No. C32461, Sep. 14, 2012
** Wikipedia
*** calculated content in 100 gr formulation SC-50-15, calculated according to the above results*.
**** Calculated content in the formulation SC-50-15 liquids only (30 ml).
***** calculation based on 34% salt concentration in Dead Sea water.

In some embodiments, the Dead Sea salt is the cosmetic preparation of mineral salts obtained from the Dead Sea sold under the tradename MINERA®, San Francisco Salt Company, San Leandro, Calif. Alternatively, the Dead Sea salt is the cosmetic grade bath salt obtained from the Dead Sea sold under the tradename AHAVA® active Dead Sea minerals, Dead Sea salt, natural Dead Sea bath salts, AHAVA dead sea laboratories Ltd. Airport City Israel.

Without intending to be limited to any particular theory, Dead Sea salts contain at least 21 minerals including magnesium, calcium, sulfur, bromine, and iodine, sodium, Zinc and potassium.

In some embodiments, the mineral salts obtained from Dead Sea comprises:

| Typical % | Range % | | |
|---|---|---|---|
| Magnesium Chloride | (MgCl2) | 33.3 | 31.0-35.0 |
| Potassium Chloride | (KCl) | 24.3 | 20.0-28.0 |
| Sodium Chloride | (NaCl) | 5.5 | 3.0-8.0 |
| Calcium Chloride | (CaCl2) | 0.2 | 0.1-0.5 |
| Bromide | (Br—) | 0.5 | 0.3-0.6 |
| Sulphates | (SO4) | 0.15 | 0.05-0.2 |
| Insolubles | | 0.03 | 0-0.3 |
| Water of Crystallization | | 36.4 | 32.0-40.0 |

In some embodiments, the mineral salts obtained from Dead Sea comprises:

| | |
|---|---|
| MgCl₂ | 33.16% |
| KCl | 27.09% |
| NaCl | 4.45% |
| CaCl₂ | 0.47% |
| H₂O | 34.81% |
| SO₄ | 0.03% |
| Br⁻ | 3590.80 ppm |

Accordingly, in some embodiments, the Dead Sea salt is replaced with a salt mixture that has a concentration of minerals including any combination of magnesium, calcium, sulfur, bromine, chloride (magnesium, potassium, sodium and/or calcium), iodine, sodium, born, zinc and potassium equivalent to Dead Sea salt.

In some embodiments, the Dead Sea salt comprises from 5 w % to 80 wt % of the composition. In some embodiments, the Dead Sea salt comprises 5 w % of the composition. In some embodiments, the Dead Sea salt comprises 10 w % of the composition. In some embodiments, the Dead Sea salt comprises 20 w % of the composition. In some embodiments, the Dead Sea salt comprises 30 w % of the composition. In some embodiments, the Dead Sea salt comprises 40 w % of the composition. In some embodiments, the Dead Sea salt comprises 50 w % of the composition. In some embodiments, the Dead Sea salt comprises 60 w % of the composition. In some embodiments, the Dead Sea salt comprises 70 w % of the composition. In some embodiments, the Dead Sea salt comprises 80 w % of the composition.

Water-Based Polymer Emulsion: In some embodiments, the water-based polymer emulsion forms a solid film on the surface of the skin suffering from a skin condition. In some embodiments, the solid film forms as the water within the composition evaporates, after the composition is applied to the skin.

In some embodiments, the water-based polymer emulsion comprises materials approved for cosmetic and/or therapeutic applications.

In some embodiments, the film is solid occlusive coating that permeable to water vapor and gasses, but impermeable to liquid water. Thus, in some embodiments, the film prevents liquid phase water transportation but allows the transportation of vapor water molecules to pass through it, without interfering with the flow of air from and to the skin.

In some embodiments, the solid film is configured to increase user compliance, and thus, the probability of prolonged and recurrent use of a composition according to some embodiments of the present invention. In some embodiments, increasing user compliance also increases the efficacy of the composition for treating the skin condition.

In some embodiments, cosmetics, such as, for example, make up may be applied to the composition, once the composition has been applied to the skin. In some embodiments, cosmetics, such as, for example, make up may be applied to the composition, once the composition has been applied to the skin, and the solid film has formed.

In some embodiments, the solid film is from 1 micron to 500 microns thick. In some embodiments, the solid film is 1 micron thick. In some embodiments, the solid film is 2 microns thick. In some embodiments, the solid film is 3 microns thick. In some embodiments, the solid film is 4 microns thick. In some embodiments, the solid film is 5 microns thick. In some embodiments, the solid film is 6 microns thick. In some embodiments, the solid film is 7 microns thick. In some embodiments, the solid film is 8 microns thick. In some embodiments, the solid film is 9 microns thick. In some embodiments, the solid film is 10 microns thick. In some embodiments, the solid film is 11 microns thick. In some embodiments, the solid film is 12 microns thick. In some embodiments, the solid film is 13 microns thick. In some embodiments, the solid film is 14 microns thick. In some embodiments, the solid film is 15 microns thick. In some embodiments, the solid film is 16 microns thick. In some embodiments, the solid film is 17 microns thick. In some embodiments, the solid film is 18 microns thick. In some embodiments, the solid film is 19 microns thick. In some embodiments, the solid film is 20 microns thick. In some embodiments, the solid film is 30 microns thick. In some embodiments, the solid film is 40 microns thick. In some embodiments, the solid film is 50 microns thick. In some embodiments, the solid film is 60 microns thick. In some embodiments, the solid film is 70 microns thick. In some embodiments, the solid film is 80 microns thick. In some embodiments, the solid film is 90 microns thick. In some embodiments, the solid film is 100 microns thick. In some embodiments, the solid film is 200 microns thick. In some embodiments, the solid film is 300 microns thick. In some embodiments, the solid film is 400 microns thick. In some embodiments, the solid film is 500 microns thick.

In some embodiments, the film is formed within 30 seconds following the application of the composition to the skin. In some embodiments, the film is formed within 60 seconds following the application of the composition to the skin. In some embodiments, the film is formed within 2 minutes following the application of the composition to the skin. In some embodiments, the film is formed within 3 minutes following the application of the composition to the skin. In some embodiments, the film is formed within 4 minutes following the application of the composition to the skin. In some embodiments, the film is formed within 5 minutes following the application of the composition to the skin. In some embodiments, the film is formed within 6 minutes following the application of the composition to the skin. In some embodiments, the film is formed within 7 minutes following the application of the composition to the skin. In some embodiments, the film is formed within 8 minutes following the application of the composition to the skin. In some embodiments, the film is formed within 9 minutes following the application of the composition to the skin. In some embodiments, the film is formed within 10 minutes following the application of the composition to the skin. In some embodiments, the film is formed within 15 minutes following the application of the composition to the skin. In some embodiments, the film is formed within 20 minutes following the application of the composition to the skin. In some embodiments, the film is formed within 25 minutes following the application of the composition to the skin. In some embodiments, the film is formed within 30 minutes following the application of the composition to the skin. In some embodiments, the film is formed within 35 minutes following the application of the composition to the skin. In some embodiments, the film is formed within 40 minutes following the application of the composition to the skin. In some embodiments, the film is formed within 45 minutes following the application of the composition to the skin. In some embodiments, the film is formed within 50 minutes following the application of the composition to the skin. In some embodiments, the film is formed within 55 minutes following the application of the composition to the skin. In some embodiments, the film is formed within 60 minutes following the application of the composition to the skin.

In some embodiments, the solid film is configured to adhere to the skin of the patient, without damaging, spoiling, or transferring to the patient's clothing or bed-linens. Furthermore, in some embodiments, the solid film is configured to remain adhered to the patient's skin and remain intact (i.e. remain as an occlusive barrier) regardless of the location applied, and the patient's physical activity. Thus, in some embodiments, the solid film is flexible. In some embodiments, the solid film is resistant to abrasion. In some embodiments, the solid film is capable of stretching. In some embodiments, the stretch modulus of the solid film is equal to the stretch modulus of skin.

In some embodiments, the solid film is configured to be a base layer for the application of makeup. In some embodiments, the solid film is configured to have a non-greasy texture.

In some embodiments, the solid film isolates the area of the skin on which the composition is applied from the external environment. In some embodiments, the solid film is configured to act as a physical barrier to microbes. In some embodiments, the area of the skin is isolated during the treatment of the skin condition. In some embodiments, the skin condition is treated by isolating the area of the skin in need of treatment from the external environment. In some embodiments, the isolation of the skin enhances the effect of a therapeutic agent incorporated in the composition.

In some embodiments, the solid film forms a concealing layer, concealing skin features, such as, for example, blemishes, redness, age spots, wrinkles, scars, inflammation, burns, pores, abrasions, and the like. In some embodiments, the composition further comprises polymeric microbeads. In some embodiments, the polymeric microbeads improve skin appearance by concealing the skin features.

In some embodiments, the water-based polymer emulsion is the polymer emulsion based on vinyl acetate, sold under the tradename VINACRYL®, Celanese Chemicals Iberica Autovia Tarragona (Spain) S.L.

In some embodiments, the water-based polymer emulsion is selected from the group consisting of: the water-based polymer emulsion is the polymer emulsion based on vinyl acetate, sold under the tradename VINACRYL® 4333, the Acrylates/Ethylhexyl Acrylate copolymer sold under the tradename KOBOGUARD 50 AMP®, Kobo Products, Inc., South Plainfield, N.J., styrene acryl polymers sold under the tradename DERMACRYL® E of Akzo nobel, polyurethane polymers sold under the tradename BAYCUSAn® C form Bayer, poly acrylic acid polymers, poly vinyl acetate polymers, poly vinyl acetate acryl copolymers, cellulosic polymers, and any combination thereof.

In some embodiments, the water-based polymer emulsion is mixed with an oil-soluble polymer configured to enhance adhesion to the skin. In some embodiments, the oil-soluble polymer is the Acrylates/Ethylhexyl Acrylate copolymer sold under the tradename KOBOGUARD 50 AMP®, Kobo Products, Inc., South Plainfield, N.J.

In some embodiments, the polymer comprises 35 wt % to 65 wt % of the water-based polymer emulsion. In some embodiments, the polymer comprises 35 wt % of the water-based polymer emulsion. In some embodiments, the polymer comprises 40 wt % of the water-based polymer emulsion. In some embodiments, the polymer comprises 45 wt % of the water-based polymer emulsion. In some embodiments, the polymer comprises 50 wt % of the water-based polymer emulsion. In some embodiments, the polymer comprises 55 wt % of the water-based polymer emulsion. In some embodiments, the polymer comprises 60 wt % of the water-based polymer emulsion. In some embodiments, the polymer comprises 65 wt % of the water-based polymer emulsion.

In some embodiments, the water-based polymer emulsion comprises from 10 wt % to 90 wt % of the composition. In some embodiments, the water-based polymer emulsion comprises 10 wt of the composition. In some embodiments, the water-based polymer emulsion comprises 20 wt of the composition. In some embodiments, the water-based polymer emulsion comprises 30 wt of the composition. In some embodiments, the water-based polymer emulsion comprises 40 wt of the composition. In some embodiments, the water-based polymer emulsion comprises 50 wt of the composition. In some embodiments, the water-based polymer emulsion comprises 60 wt of the composition. In some embodiments, the water-based polymer emulsion comprises 70 wt of the composition. In some embodiments, the water-based polymer emulsion comprises 80 wt of the composition. In some embodiments, the water-based polymer emulsion comprises 90 wt of the composition.

In some embodiments, the composition further comprises a wetting and dispersing additive up to 20 wt % of the composition. In some embodiments, the wetting and dispersing additive comprises 0.1 wt % of the composition. In some embodiments, the wetting and dispersing additive comprises 0.2 wt % of the composition. In some embodiments, the wetting and dispersing additive comprises 0.3 wt % of the composition. In some embodiments, the wetting and dispersing additive comprises 0.4 wt % of the composition. In some embodiments, the wetting and dispersing additive comprises 0.5 wt % of the composition. In some embodiments, the wetting and dispersing additive comprises 1 wt % of the composition. In some embodiments, the wetting and dispersing additive comprises 2 wt % of the composition. In some embodiments, the wetting and dispersing additive comprises 3 wt % of the composition. In some embodiments, the wetting and dispersing additive comprises 4 wt % of the composition. In some embodiments, the wetting and dispersing additive comprises 5 wt % of the composition. In some embodiments, the wetting and dispersing additive comprises 6 wt % of the composition. In some embodiments, the wetting and dispersing additive comprises 7 wt % of the composition. In some embodiments, the wetting and dispersing additive comprises 8 wt % of the composition. In some embodiments, the wetting and dispersing additive comprises 9 wt % of the composition. In some embodiments, the wetting and dispersing additive comprises 10 wt % of the composition. In some embodiments, the wetting and dispersing additive comprises 12 wt % of the composition. In some embodiments, the wetting and dispersing additive comprises 14 wt % of the composition. In some embodiments, the wetting and dispersing additive comprises 16 wt % of the composition. In some embodiments, the wetting and dispersing additive comprises 18 wt % of the composition. In some embodiments, the wetting and dispersing additive comprises 20 wt % of the composition.

In some embodiments, the composition further comprises a wetting and dispersing additive up to 20 wt % of the composition. In some embodiments, the wetting and dispersing additive is a wetting and dispersing additive for water-based coatings. In some embodiments, the wetting and dispersing additive is a wetting and dispersing additive for printing inks. In some embodiments, the wetting and dispersing additive is a wetting and dispersing additive for cosmetic preparations. In some embodiments, the wetting and dispersing additive is the wetting and dispersing additive sold under the tradename DISPERBYK®. In some embodiments, the wetting and dispersing additive is the wetting and dispersing additive sold under the tradename DYNASYLAN® 4150. In some embodiments, the wetting and dispersing additive is the wetting and dispersing additive sold under the tradename DIPERSUN DSP-W90®.

In some embodiments, the composition further comprises a rheology modifier up to 30 wt % of the composition. In some embodiments, the rheology modifier comprises 0.1 wt % of the composition. In some embodiments, the rheology modifier comprises 0.2 wt % of the composition. In some embodiments, the rheology modifier comprises 0.3 wt % of the composition. In some embodiments, the rheology modifier comprises 0.4 wt % of the composition. In some embodiments, the rheology modifier comprises 0.5 wt % of the composition. In some embodiments, the rheology modifier comprises 1 wt % of the composition. In some embodiments, the rheology modifier comprises 2 wt % of the composition. In some embodiments, the rheology modifier comprises 3 wt % of the composition. In some embodiments, the rheology modifier comprises 4 wt % of the composition. In some embodiments, the rheology modifier comprises 5 wt % of the composition. In some embodiments, the rheology modifier comprises 6 wt % of the composition. In some embodiments, the rheology modifier comprises 7 wt % of the composition. In some embodiments, the rheology modifier comprises 8 wt % of the composition. In some embodiments, the rheology modifier comprises 9 wt % of the composition. In some embodiments, the rheology modifier comprises 10 wt % of the composition. In some embodiments, the rheology modifier comprises 12 wt % of the composition. In some embodiments, the rheology modifier comprises 14 wt % of the composition. In some embodiments, the rheology modifier comprises 16 wt % of the composition. In some embodiments, the rheology modifier comprises 18 wt % of the composition. In some embodiments, the rheology modifier comprises 20 wt % of the composition. In some embodiments, the rheology modifier comprises 22 wt % of the composition. In some embodiments, the rheology modifier comprises 24 wt % of the composition. In some embodiments, the rheology modifier comprises 26 wt % of the composition. In some embodiments, the rheology modifier comprises 28 wt % of the composition. In some embodiments, the rheology modifier comprises 30 wt % of the composition.

In some embodiments, the rheology modifier is selected from the group consisting of the non-ionic thickener based on polyurethane sold under the tradename TEGO® Rheo8600, the polymer emulsion comprising a mixture of a polyurethane alkylate co polymer with fatty alcohols sold under the tradename LUVIGEL®, BASF Care Creations, the rheology modifier sold under the tradename LUVIGEL® Star AT3, sodium poly acrylate, poly acrylic acid, poly carbamide, isoparffin, ethylhexylstearate, cellulosic polymers, such as, for example, hydroxy ethyl cellulose (HEC), carboxy methyl cellulose (CMC), hydroxy propyl methyl cellulose (HPMC), polyvinylpyrrolodone homopolymers (PVP) such as, for example, the PVP sold under the tradename LUVISKOL® K30.

In some embodiments, the rheology modifier has a dynamic viscosity, when measured at 25° C. of approximately 30,000 mPas.

In some embodiments, the water-based emulsion has a low Tg (less than 0° C.), and thus is flexible at room and skin surface temperature. As a result, in some embodiments, the solid film, when formed, expands and contracts with the skin movement, and will prevent cracks and pealing of the solid film.

In some embodiments, the viscosity of the composition is configured to promote skin adhesion and skin coverage. In some embodiments, the rheology modifier is added in an amount sufficient to achieve the required viscosity of the composition. In some embodiments, the viscosity of the composition is from 4000 to 23000 mPas.

In some embodiments, the viscosity of the composition is from 500 to 1,000,000 mPas. In some embodiments, the viscosity of the composition is 500 mPas. In some embodiments, the viscosity of the composition is 1000 mPas. In some embodiments, the viscosity of the composition is 1,500 mPas. In some embodiments, the viscosity of the composition is 2,000 mPas. In some embodiments, the viscosity of the composition is 4,000 mPas. In some embodiments, the viscosity of the composition is 6,000 mPas. In some embodiments, the viscosity of the composition is 8,000 mPas. In some embodiments, the viscosity of the composition is 10,000 mPas. In some embodiments, the viscosity of the composition is 12,000 mPas. In some embodiments, the viscosity of the composition is 14,000 mPas. In some embodiments, the viscosity of the composition is 15,000 mPas. In some embodiments, the viscosity of the composition is 16,000 mPas. In some embodiments, the viscosity of the composition is 17,000 mPas. In some embodiments, the viscosity of the composition is 18,000 mPas. In some embodiments, the viscosity of the composition is 19,000 mPas. In some embodiments, the viscosity of the composition is 20,000 mPas. In some embodiments, the viscosity of the composition is 21,000 mPas. In some embodiments, the viscosity of the composition is 22,000 mPas. In some embodiments, the viscosity of the composition is 23,000 mPas. In some embodiments, the viscosity of the composition is 24,000 mPas. In some embodiments, the viscosity of the composition is 25,000 mPas. In some embodiments, the viscosity of the composition is 50,000 mPas. In some embodiments, the viscosity of the composition is 100,000 mPas. In some embodiments, the viscosity of the composition is 200,000 mPas. In some embodiments, the viscosity of the composition is 300,000 mPas. In some embodiments, the viscosity of the composition is 400,000 mPas. In some embodiments, the viscosity of the composition is 500,000 mPas. In some embodiments, the viscosity of the composition is 600,000 mPas. In some embodiments, the viscosity of the composition is 700,000 mPas. In some embodiments, the viscosity of the composition is 800,000 mPas. In some embodiments, the viscosity of the composition is 900,000 mPas. In some embodiments, the viscosity of the composition is 1,000,000 mPas.

In some embodiments, the composition further comprises a deafoamer up to 8 wt % of the composition. In some embodiments, the defoamer comprises 0.1 wt % of the composition. In some embodiments, the defoamer comprises 0.2 wt % of the composition. In some embodiments, the defoamer comprises 0.3 wt % of the composition. In some embodiments, the defoamer comprises 0.4 wt % of the composition. In some embodiments, the defoamer comprises 0.5 wt % of the composition. In some embodiments, the defoamer comprises 1 wt % of the composition. In some embodiments, the defoamer comprises 2 wt % of the composition. In some embodiments, the defoamer comprises 3 wt % of the composition. In some embodiments, the defoamer comprises 4 wt % of the composition. In some embodiments, the defoamer comprises 5 wt % of the composition. In some embodiments, the defoamer comprises 6 wt % of the composition. In some embodiments, the defoamer comprises 7 wt % of the composition. In some embodiments, the defoamer comprises 8 wt % of the composition.

In some embodiments, the defoamer is the defoamer sold under the tradename TEGO® Foamex825. In some embodiments, the defoamer is the defomaer sold under the tradename XIAMETER® AFE 1510. In some embodiments, the defoamer is the defomaer sold under the tradename BC SIMETHICONE ANTIFOAMER PD30S.

In one embodiment, the composition further comprises water up to 90 wt % of the composition. In some embodiments, the water comprises 0.1 wt % of the composition. In some embodiments, the water comprises 0.2 wt % of the composition. In some embodiments, the water comprises 0.3 wt % of the composition. In some embodiments, the water comprises 0.4 wt % of the composition. In some embodiments, the water comprises 0.5 wt % of the composition. In some embodiments, the water comprises 1 wt % of the composition. In some embodiments, the water comprises 2 wt % of the composition. In some embodiments, the water comprises 3 wt % of the composition. In some embodiments, the water comprises 4 wt % of the composition. In some embodiments, the water comprises 5 wt % of the composition. In some embodiments, the water comprises 6 wt % of the composition. In some embodiments, the water comprises 7 wt % of the composition. In some embodiments, the water comprises 8 wt % of the composition. In some embodiments, the water comprises 9 wt % of the composition. In some embodiments, the water comprises 10 wt % of the composition. In some embodiments, the water comprises 12 wt % of the composition. In some embodiments, the water comprises 14 wt % of the composition. In some embodiments, the water comprises 16 wt % of the composition. In some embodiments, the water comprises 18 wt % of the composition. In some embodiments, the water comprises 20 wt % of the composition. In some embodiments, the water comprises 22 wt % of the composition. In some embodiments, the water comprises 24 wt % of the composition. In some embodiments, the water comprises 26 wt % of the composition. In some embodiments, the water comprises 28 wt % of the composition. In some embodiments, the water comprises 30 wt % of the composition. In some embodiments, the water comprises 40 wt % of the composition. In some embodiments, the water comprises 50 wt % of the composition. In some embodiments, the water comprises 60 wt % of the composition. In some embodiments, the water comprises 70 wt % of the composition. In some embodiments, the water comprises 80 wt % of the composition. In some embodiments, the water comprises 90 wt % of the composition.

In some embodiments, the composition further comprises at least one additional component selected from the group consisting of: a humectant, an alcohol, an adhesion promoter, a lubricant, a softening agent, a fragrance, and a therapeutic agent.

In some embodiments, the humectant absorbs and holds water. Thus, without intending to be limited to any particular theory, once the composition is applied to the skin, the humectant slows the drying process. In some embodiments, a slower drying process allows the faster onset, higherinitial effective concentration and longer availability and penetration period for the dissolved active ions into the skin pores and wounds.

In some embodiments, the humectant prolongs the drying time of the water-based polymer emulsion. In some embodiments, the prolonged drying time of the water-based polymer emulsion lengthens the time required for the composition to form a film. In some embodiments, the humectant is added in an amount sufficient to produce a water-based polymer solution with the desired drying time. In some embodiments, the humectant comprises a glycol.

In some embodiments, the alcohol shortens the drying time of the water-based polymer emulsion. In some embodiments, the shortened drying time of the water-based polymer emulsion reduces the time required for the composition to form a film. In some embodiments, the alcohol is added in an amount sufficient to produce a water-based polymer solution with the desired drying time. In some embodiments, the alcohol is selected from the group consisting of ethanol, and iso-propyl alcohol.

In some embodiments, the water-based polymer emulsion is mixed with an oil-soluble polymer configured to enhance adhesion to the skin. Suitable oil-soluble polymers include, but are not limited to: the Acrylates/Ethylhexyl Acrylatecopolymer sold under the tradename KOBOGUARD 50 AMP®, Kobo Products, Inc., South Plainfield, N.J., the polymer sold under the tradename DERMACRYL® AQF, the polymer sold under the tradename DERMACRYL® E from AkzoNobel, the polyurethane polymers sold under the tradenames AVALURE AC 120, AVALURE AC 210 or AVALURE UR 450 from Lubrizol, the acrylate polymer sold under the tradenames and Acrylates, the polymers sold under the tradename SENSIENT, and laolin derivatives.

In some embodiment, the lubricant is configured to provide a soft feel to the composition when applied to the skin. In some embodiments, the lubricant is a wax. In some embodiments, the lubricant is an oil.

In some embodiments, the composition further comprises a surfactant. In some embodiments, the surfactant stabilizes the components of the composition and aids in the formation of a homogeneous composition. In some embodiments, the surfactant is a non-ionic surfactant. In some embodiments, the surfactant is a cationic surfactant. In some embodiments, the surfactant is an anionic surfactant. In some embodiments, the surfactant is a poly anionic surfactant. In some embodiments, the surfactant is a poly cationic surfactant. In some embodiments, the surfactant is a quaternary amine. In some embodiments, the surfactant is an ethoxylated alcohol. In some embodiments, the surfactant is a nonylphenol. In some embodiments, the surfactant is a polyquaternium surfactant.

In some embodiments, the composition further comprises a pigment. In some embodiments, the pigment is a cosmetic pigment. In some embodiments, the pigment is a mineral pigment. In some embodiments, the pigment is an oxide pigment. In some embodiments, the pigment is natural. In some embodiments, the pigment is synthetic. In some embodiments, the pigment is a fine particle. In some embodiments, the pigment is based on poly methyl metacrylate.

Suitable mineral pigments include, but are not limited to iron oxide yellow, iron oxide red, iron oxide brown, and iron oxide black.

Suitable oxide pigments include, but are not limited to titanium dioxide, cobalt(II) oxide, and aluminium oxide.

In some embodiments, the pigment is up to 20 wt % of the composition. In some embodiments, the pigment comprises 0.1 wt % of the composition. In some embodiments, the pigment comprises 0.2 wt % of the composition. In some embodiments, the pigment comprises 0.3 wt % of the composition. In some embodiments, the pigment comprises 0.4 wt % of the composition. In some embodiments, the pigment comprises 0.5 wt % of the composition. In some embodiments, the pigment comprises 1 wt % of the composition. In some embodiments, the pigment comprises 2 wt % of the composition. In some embodiments, the pigment comprises 3 wt % of the composition. In some embodiments, the pigment comprises 4 wt % of the composition. In some embodiments, the pigment comprises 5 wt % of the composition. In some embodiments, the pigment comprises 6 wt % of the composition. In some embodiments, the pigment comprises 7 wt % of the composition. In some embodiments, the pigment comprises 8 wt % of the composition. In some embodiments, the pigment comprises 9 wt % of the composition. In some embodiments, the pigment comprises 10 wt % of the composition. In some embodiments, the pigment comprises 12 wt % of the composition. In some embodiments, the pigment comprises 14 wt % of the composition. In some embodiments, the pigment comprises 16 wt % of the composition. In some embodiments, the pigment comprises 18 wt % of the composition. In some embodiments, the pigment comprises 20 wt % of the composition.

In some embodiments, the composition is transparent.

In some embodiments, the composition further comprises solid particles. In some embodiments, the solid particles are fillers. In some embodiments, the filler is added to the composition in an amount sufficient to result in good adhesion of the composition on the skin, without blocking or encapsulating any of the active components. In some embodiments, the filler comprises 15 wt % to 60 wt %, relative to other solid particles in the composition

In some embodiments, the solid particles conceal skin features. In some embodiments, the solid particles fill skin features. In some embodiments, the solid particles are pigments. In some embodiments, the solid particles are selected from the group consisting of: CaCO₃, mica, MgO, dolomite, talc, polymeric particles, SiO₂, and clay.

In some embodiments, the composition further comprises a smectite clay up to 30 wt % of the composition. In some embodiments, the smectite clay comprises 0.1 wt % of the composition. In some embodiments, the smectite clay comprises 0.2 wt % of the composition. In some embodiments, the smectite clay comprises 0.3 wt % of the composition. In some embodiments, the smectite clay comprises 0.4 wt % of the composition. In some embodiments, the smectite clay comprises 0.5 wt % of the composition. In some embodiments, the smectite clay comprises 1 wt % of the composition. In some embodiments, the smectite clay comprises 2 wt % of the composition. In some embodiments, the smectite clay comprises 3 wt % of the composition. In some embodiments, the smectite clay comprises 4 wt % of the composition. In some embodiments, the smectite clay comprises 5 wt % of the composition. In some embodiments, the smectite clay comprises 6 wt % of the composition. In some embodiments, the smectite clay comprises 7 wt % of the composition. In some embodiments, the smectite clay comprises 8 wt % of the composition. In some embodiments, the smectite clay comprises 9 wt % of the composition. In some embodiments, the smectite clay comprises 10 wt % of the composition. In some embodiments, the smectite clay comprises 12 wt % of the composition. In some embodiments, the smectite clay comprises 14 wt % of the composition. In some embodiments, the smectite clay comprises 16 wt % of the composition. In some embodiments, the smectite clay comprises 18 wt % of the composition. In some embodiments, the smectite clay comprises 20 wt % of the composition. In some embodiments, the smectite clay comprises 22 wt % of the composition. In some embodiments, the smectite clay comprises 24 wt % of the composition. In some embodiments, the smectite clay comprises 26 wt % of the composition. In some embodiments, the smectite clay comprises 28 wt % of the composition. In some embodiments, the smectite clay comprises 30 wt % of the composition.

In some embodiments, the smectite clay is the smectite clay sold under the tradename BENTONE® EW.

In some embodiments, the fragrance is up to 5 wt % of the composition.

In some embodiments, the therapeutic agent is an anti-bacterial agent. In some embodiments, the antibacterial agent is salicylic acid. In some embodiments, the salicylic acid is up to 10 wt % of the composition. In some embodiments, the salicylic acid comprises 0.1 wt % of the composition. In some embodiments, the salicylic acid comprises 0.2 wt % of the composition. In some embodiments, the salicylic acid comprises 0.3 wt % of the composition. In some embodiments, the salicylic acid comprises 0.4 wt % of the composition. In some embodiments, the salicylic acid comprises 0.5 wt % of the composition. In some embodiments, the salicylic acid comprises 1 wt % of the composition. In some embodiments, the salicylic acid comprises 2 wt % of the composition. In some embodiments, the salicylic acid comprises 3 wt % of the composition. In some embodiments, the salicylic acid comprises 4 wt % of the composition. In some embodiments, the salicylic acid comprises 5 wt % of the composition. In some embodiments, the salicylic acid comprises 6 wt % of the composition. In some embodiments, the salicylic acid comprises 7 wt % of the composition. In some embodiments, the salicylic acid comprises 8 wt % of the composition. In some embodiments, the salicylic acid comprises 9 wt % of the composition. In some embodiments, the salicylic acid comprises 10 wt % of the composition.

In some embodiments, the therapeutic agent is a component of the extracelluar matrix. In some embodiments, the component of the extracellular matrix is added in an amount effective to treat wrinkles. In some embodiments, the component of the extracelluar matrix treats wrinkles by swelling the extracellular matrix. In some embodiments, the component of the extracellular matrix is hyaluronic acid.

In some embodiments, the hyaluronic acid is up to 10 wt % of the composition. In some embodiments, the hyaluronic acid comprises 0.1 wt % of the composition. In some embodiments, the hyaluronic acid comprises 0.2 wt % of the composition. In some embodiments, the hyaluronic acid comprises 0.3 wt % of the composition. In some embodiments, the hyaluronic acid comprises 0.4 wt % of the composition. In some embodiments, the hyaluronic acid comprises 0.5 wt % of the composition. In some embodiments, the hyaluronic acid comprises 1 wt % of the composition. In some embodiments, the hyaluronic acid comprises 2 wt % of the composition. In some embodiments, the hyaluronic acid comprises 3 wt % of the composition. In some embodiments, the hyaluronic acid comprises 4 wt % of the composition. In some embodiments, the hyaluronic acid comprises 5 wt % of the composition. In some embodiments, the hyaluronic acid comprises 6 wt % of the composition. In some embodiments, the hyaluronic acid comprises 7 wt % of the composition. In some embodiments, the hyaluronic acid comprises 8 wt % of the composition. In some embodiments, the hyaluronic acid comprises 9 wt % of the composition. In some embodiments, the hyaluronic acid comprises 10 wt % of the composition.

Other examples of therapeutic agents include, but are not limited to, antibiotics for topical application, such as, for example, chloramphenicol, Mupirocin, other aanitiicrobial agents, such as sulfur, sulfur derivatives, sulphates, zinc, zinc oxide, magnesium, magnesuim oxide, magnesium chloride, titanium dioxide, chloride, and ammonium bituminosulfonate. These agents may comprise up to 15% of the composition.

In some embodiments, the composition is configured to be removed by washing with water.

Compositions According to Some Embodiments of the Present Invention. In some embodiments, the composition is formulated as a cream. In some embodiments, the cream is water-based, and lacks emulsified oils and/or hydrophobic skin penetrating components. Without intending to be limited to any particular theory, the salts and active components of the cream composition according to some embodiments of the present invention exist in their ionic (i.e. in solution), or, alternatively, in a super-saturated state. Again, without intending to be limited to any particular theory, once the composition is applied to the skin, the salts will penetrate into the target area.

In some embodiments, the composition is formulated as a sol-gel. In some embodiments, the composition is incorporated into a carrier, such as, for example, a bandage, or, alternatively, an item of clothing.

In some embodiments, the composition comprises the composition set forth in Table 1.

TABLE 1

| material | gr | % w | comments |
|---|---|---|---|
| Dead sea salt | 100 | 49.3 | |
| Vinacryl 4333 | 83 | 40.93 | |
| Luvigel Star AT3 | 12.2 | 6.02 | |
| Benton EW | 3 | 1.48 | |
| Luviskol K30 | 1.8 | 0.89 | |
| Salicylic Acid | 1 | 0.493 | |
| Florma paste 15653 | 1 | 0.493 | |
| Tego foamex 825 | 0.8 | 0.394 | |
| Total | 202.8 | 100 | |

| | |
|---|---|
| Salt %** | 70.7 |
| pH | 3.66 |
| Viscosity (cps)* | 7,130 |
| Consistency and | Separation after 4 weeks. No salt |
| shelf life stability. | recrystallization and no skin formation on top. |
| Resistance Ω | 1230 |
| Ohm*** | |
| Subjects tested | Active. A little irritant. |
| comments | |

*Brookfield spindle 6, 60 rpm
**Salt w/liquid w in the formulation
***measured with SAKAL DT-823

In some embodiments, the composition comprises the composition set forth in Table 2.

TABLE 2

| material | gr | % w | comments |
|---|---|---|---|
| Dead sea salt | 100 | 50 | |
| Vinacryl 4333 | 80.3 | 39 | |
| Luvigel Star AT3 | 12.2 | 6.1 | |
| Benton EW | 3 | 1.5 | |
| Luviskol K30 | 1.8 | 0.9 | |
| Salicylic Acid | 0.9 | 0.45 | |
| Florma paste 15653 | 1 | 0.5 | |
| Tego foamex 825 | 0.8 | 0.4 | |
| Total | 200 | 100 | |

| | |
|---|---|
| Salt %** | 72 |
| pH | 3.64 |
| Viscosity* (cps) | 10,200 |
| Consistency and | Separation after 4 weeks. No salt |
| shelf life stability. | recrystallization and no skin formation on top. |
| Resistance Ω | 1248 |
| Ohm*** | |
| Subjects tested | Active. Leave visible white spots after drying |
| comments | on skin, which is not easily washed away. |

*Brookfield spindle 6, 60 rpm
**Salt w/liquid w in the formulation
***measured with SAKAL DT-823

In some embodiments, the composition comprises the composition set forth in Table 3.

TABLE 3

| material | gr | % w | comments |
|---|---|---|---|
| Dead sea salt | 100 | 50 | |
| Kobogaurd AMP50 | 89.3 | 44.6 | |
| Luvigel Star AT3 | 6.6 | 3.3 | |
| Dynasylan4150 | 3.3 | 1.65 | |
| Tego foamex 825 | 0.8 | 0.4 | |
| Total | 200 | 100 | |

| | |
|---|---|
| Salt %** | 70.9 |
| pH | 4.85 |
| Viscosity* (cps) | 10,940 |
| Consistency and | No separation after 4 weeks. No salt |
| shelf life stability. | recrystallization and no skin formation on top. |
| Resistance Ω | 1360 |
| Ohm*** | |
| Subjects tested | Active. After drying leaves a transparent |
| comments | flexible film on skin |

*Brookfield spindle 6, 60 rpm
**Salt w/liquid w in the formulation
***measured with SAKAL DT-823

In some embodiments, the composition comprises the composition set forth in Table 4.

TABLE 4

| material | gr | % w | comments |
|---|---|---|---|
| Dead sea salt | 105.3 | 52.65 | |
| Kobogaurd AMP50 | 79 | 39.5 | |
| Luvigel Star AT3 | 6.1 | 3.05 | |
| Dynasylan4150 | 4 | 2 | |
| Benton EW | 3 | 1.5 | |
| Luviskol K30 | 1.8 | 0.9 | |
| Tego foamex 825 | 0.8 | 0.4 | |
| Total | 200 | 100 | |

| | |
|---|---|
| Salt %** | 79.5 |
| pH | 4.93 |
| Viscosity* (cps) | 10,540 |
| Consistency and | Separation starts after 3 weeks. No salt |
| shelf life stability. | recrystallization and no skin formation on top. |
| Resistance Ω | 1145 |
| Ohm*** | |
| Subjects tested | Active. After drying leaves a transparent |
| comments | flexible film on skin. |
| | Irritant for sensitive skin. |

*Brookfield spindle 6, 60 rpm
**Salt w/liquid w in the formulation
***measured with SAKAL DT-823

In some embodiments, the composition comprises the composition set forth in Table 5.

TABLE 5

| material | gr | % w | comments |
|---|---|---|---|
| Dead sea salt | 105.6 | 52.8 | |
| Kobogaurd AMP50 | 82.6 | 41.3 | |
| Luvigel Star AT3 | 6.1 | 3.05 | |
| Dynasylan4 150 | 3.1 | 1.55 | |
| Luviskol K30 | 1.8 | 0.9 | |
| AFE 1510 | 0.8 | 0.4 | |
| Total | 200 | 100 | |

| | |
|---|---|
| Salt %** | 74.6 |
| pH | 4.78 |
| Viscosity* (cps) | 5,200 |
| Consistency and | Separation starts after 2 weeks. No salt |
| shelf life stability. | recrystallization and no skin formation on top. |
| Resistance Ω | 1210 |
| Ohm*** | |
| Subjects tested | Active. After drying leaves a transparent |
| comments | flexible film on skin. |
| | Irritant for sensitive skin. |

*Brookfield spindle 6, 60 rpm
**Salt w/liquid w in the formulation
***measured with SAKAL DT-823

In some embodiments, the composition comprises the composition set forth in Table 6.

TABLE 6

| material | gr | % w | comments |
|---|---|---|---|
| Dead sea salt | 105.6 | 52.85 | Dried salt |
| Kobogaurd AMP50 | 83.3 | 41.60 | |
| Luvigel Star AT3 | 4.7 | 2.35 | |
| Dynasylan4 150 | 3.1 | 1.55 | |
| Luviskol K30 | 2 | 1 | |
| AFE 1510 | 1.3 | 0.65 | |
| Total | 199.8 | 100 | |

| | |
|---|---|
| Salt %** | 70.9 |
| pH | 4.79 |
| Viscosity* (cps) | 9,300 |
| Consistency and | Separation starts after 3 weeks. No salt |
| shelf life stability. | recrystallization and no skin formation on top. |
| Resistance Ω | 1215 |
| Ohm*** | |
| Subjects tested | Active. After drying leaves a transparent |
| comments | flexible film on skin. |
| | Irritant for sensitive skin. |

*Brookfield spindle 7, 60 rpm
**Salt w/liquid w in the formulation
***measured with SAKAL DT-823

In some embodiments, the composition comprises the composition set forth in Table 7.

TABLE 7

| material | gr | % w | comments |
|---|---|---|---|
| Dead sea salt | 105.6 | 52.8 | Dried salt |
| Kobogaurd AMP50 | 80.6 | 40.3 | |
| Luvigel Star AT3 | 5.2 | 2.6 | |
| Dynasylan4 150 | 4 | 2 | |
| Luviskol K30 | 1.8 | 0.9 | |
| Benton EW | 1.5 | 0.75 | |
| AFE 1510 | 1.3 | 0.65 | |
| Total | 200 | 100 | |

| | |
|---|---|
| Salt % | 76.6 |
| pH | 4.83 |
| Viscosity* (cps) | 4,940 |
| Consistency and | Separation starts after 3 weeks. No salt |
| shelf life stability. | recrystallization and no skin formation on top. |
| Resistance Ω | 1160 |
| Ohm*** | |
| Subjects tested | Active. After drying leaves a transparent |
| comments | flexible film on skin. |
| | Irritant for sensitive skin. |

*Brookfield spindle 6, 60 rpm
** Salt w/liquid w in the formulation
***measured with SAKAL DT-823

In some embodiments, the composition comprises the composition set forth in Table 8.

TABLE 8

| material | gr | % w | comments |
|---|---|---|---|
| Dead sea salt | 105 | 52.5 | Dried salt |
| Kobogaurd AMP50 | 80.1 | 40.05 | |
| Luvigel Star AT3 | 6.1 | 3.05 | |
| Dynasylan4 150 | 4 | 2 | |
| Luviskol K30 | 1.5 | 0.75 | |
| Benton EW | 2 | 1 | |
| AFE 1510 | 1.3 | 0.65 | |
| Total | 200 | 100 | |

| | |
|---|---|
| Salt % | 76.1 |
| pH | 4.86 |
| Viscosity* (cps) | 10,930 |
| Consistency and | Separation starts after 10 days. No salt |
| shelf life stability. | recrystallization and no skin formation on top. |
| Resistance Ohm*** | 1180 |
| Subjects tested | Active. After drying leaves a transparent |
| comments | flexible film on skin. |
| | Irritant for sensitive skin. |

*Brookfield spindle 6, 60 rpm
** Salt w/liquid w in the formulation
***measured with SAKAL DT-823

In some embodiments, the composition comprises the composition set forth in Table 9.

TABLE 9

| material | gr | % w | comments |
|---|---|---|---|
| Dead sea salt | 104 | 52 | Dried salt |
| Kobogaurd AMP50 | 82 | 41 | |
| Luvigel Star AT3 | 6 | 3 | |
| Dynasylan4 150 | 4 | 2 | |
| Luviskol K30 | 1.8 | 0.9 | |
| Benton EW | 1.5 | 0.75 | |
| AFE 1510 | 0.7 | 0.35 | |
| Total | 200 | 100 | |

| | |
|---|---|
| Salt % | 75.6 |
| pH | 4.91 |
| Viscosity* (cps) | 12,400 |
| Consistency and | Separation starts after 8 days. No salt |
| shelf life stability. | recrystallization and no skin formation on top. |
| Resistance Ohm*** | 1140 |
| Subjects tested | Active. After drying leaves a transparent |
| comments | flexible film on skin. |
| | Irritant for sensitive skin. |

*Brookfield spindle 6, 60 rpm
** Salt w/liquid w in the formulation
***measured with SAKAL DT-823

In some embodiments, the composition comprises the composition set forth in Table 10.

TABLE 10

| material | gr | % w | comments |
|---|---|---|---|
| Dead sea salt | 100 | 47 | |
| Kobogaurd AMP50 | 92 | 43.3 | |
| Luvigel Star AT3 | 10.0 | 4.8 | |
| Dynasylan4 150 | 6 | 2.8 | |
| Benton EW | 3 | 1.41 | |
| AFE 1510 | 0.8 | 0.38 | |
| Distil water | 0.3 | 0.14 | |
| Luviskol K30 | 0.2 | 0.1 | |
| Total | 212.3 | 100 | |

| | |
|---|---|
| Salt %** | 70.3 |
| pH | 5.04 |
| Viscosity* (cps) | 22,400 |
| Consistency and | No separation. No salt |
| shelf life stability. | recrystallization and no skin formation on top. |
| Resistance Ohm*** | 1120 |
| Subjects tested | Active. After drying leaves a transparent |
| comments | flexible film on skin. |
| | Slightly to not Irritant. |

*Brookfield spindle 6, 60 rpm
**Salt w/liquid w in the formulation
***measured with SAKAL DT-823

In some embodiments, the composition comprises the composition set forth in Table 11.

TABLE 11

| material | gr | % w | comments |
|---|---|---|---|
| Dead sea salt | 100 | 48.4 | |
| Kobogaurd AMP50 | 87 | 42.11 | |
| Luvigel Star AT3 | 8.8 | 4.25 | |
| Dynasylan4 150 | 7 | 3.4 | |
| Benton EW | 3 | 1.45 | |
| AFE 1510 | 0.8 | 0.4 | |
| Total | 206.6 | 100 | |

| | |
|---|---|
| Salt % ** | 72.4 |
| pH | 4.94 |
| Viscosity* (cps) | 10,600 |
| Consistency and | Separation starts after 10 days. No salt |
| shelf life stability. | recrystallization and no skin formation on top. |
| Resistance Ohm*** | 1128 |
| Subjects tested | Active. After drying leaves a transparent |
| comments | flexible film on skin. |
| | Irritant for sensitive skin. |

*Brookfield spindle 6, 60 rpm
** Salt w/liquid w in the formulation
***measured with SAKAL DT-823

### Methods to Manufacture the Composition According to Some Embodiments of the Present Invention

In some embodiments, the composition is formed by adding the wetting and dispersing additive to the solution of the water-based polymer emulsion, and mixing to ensure adequate dispersion. Without intending to be limited to any particular theory, the wetting and dispersing additive increases the stability of the water-based polymer emulsion before the addition of high salt, by adding a layer of stabilizing molecules to the emulsion.

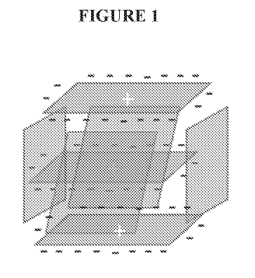

After the wetting and dispersing additive is added and dispersed, in some embodiments, the smectite clay Benton EW is added and well dispersed at high shear rate (greater than 1000 [1\s] for more than 20 min. Without intending to be limited to any particular theory, the addition of the wetting and dispersing additive enables the later opening of the clay, such as Benton EW thickener to form a “card stack” matrix structure (see FIG. 1).

Without intending to be limited to any particular theory, the addition of the components in the order described above prevents the syneresis of the formulation.

Next, in some embodiments, Dead Sea salt, having a particle size from 1-500 microns is added slowly, at 100 [1\s] shear rate and at approximately 10 g\sec, to avoid mechanical shear and allow chemical stress dissipation of the salt on the emulsion.

Next, the additional components, such as, for example, salicylic acid, pigments and the rheology modifier are added. In some embodiments, the rheology modifier is added after the salt to avoid breakdown of the formulation.

The formulations according to some embodiments of the present invention were tested and found to be stable for at least 12 weeks, with no changes in appearance, no change in odor, viscosity, or pH.

In some embodiments, the composition results in a high concentration of mineral salts at the skin surface, without altering the performance of the solid film.

In some embodiments, the solid film is a reservoir of ions of the mineral salts, configured to deliver the ions to the skin of the patient.

In some embodiments, there is a layer of moisture between the skin and the solid film. In some embodiments, the moisture is sweat. Without intending to be limited to any particular theory, layer of sweat and water between the solid film and the skin in which the mineral salts carried by the solid film is dissolved or super saturated (thermodynamically dependent) and is diffused according to Fick's law from high to low concentration gradient (of the ion\solute) at the target zone.

Without intending to be limited by any particular theory, the mineral salt reservoir establishes the driving force for the diffusion mechanism delivery of the ion\solute to the skin\target zone and the suppression of the osmotic mechanism (water flux from the skin to the film), resulting in a continuous and stable ion\solute flux directed into the skin.

In some embodiments, the concentration of mineral salts in the solid film is from 30 wt % of the solid film to 70 wt % of the solid film. In some embodiments, the concentration of mineral salts in the solid film is 30 wt %. In some embodiments, the concentration of mineral salts in the solid film is 40 wt %. In some embodiments, the concentration of mineral salts in the solid film is 50 wt %. In some embodiments, the concentration of mineral salts in the solid film is 60 wt %. In some embodiments, the concentration of mineral salts in the solid film is 70 wt %.

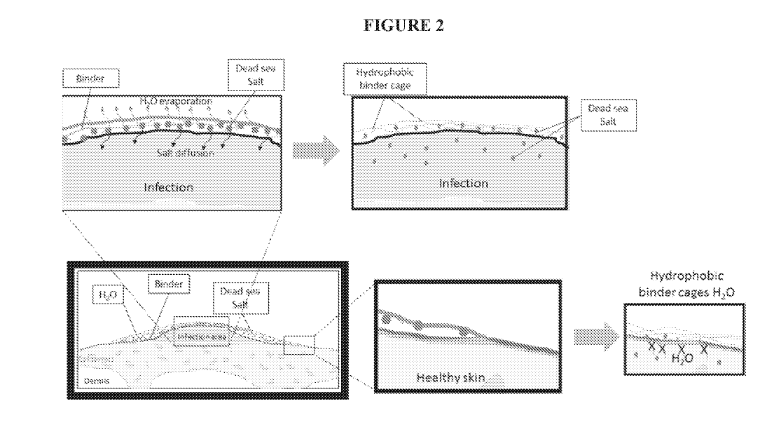

The following is a theory regarding how the solid film of the present invention acts as a delivery system. This theory does not limit the invention as discussed herein. The thermodynamic driving forces controlling the flux of salts and active components flux the target area of the skin zone will be described below, and in FIG. 2.

Upon application of the composition to the skin surface, the free water within the composition can penetrate with the dissolved and non-dissolved active components into skin beneath the applied composition, or evaporate without the active components to the surrounding air. The water evaporation generates a top dry surface layer on the composition, which is a result of the film formation process of the hydrophobic binder.

As the drying process progresses, the composition dries off and creates a hydrophobic film on the skin or wound. This film is a salt containing polymer matrix stably adhered to the surface. The film of the dry hydrophobic binder seals the wound, preventing liquid water transportation and penetration of contaminants, but allows the transport of vapor water molecules/moisture through the film, and also prevents the composition from peeling off the skin.

The hydrophobic film maintains valuable moisture produced by the skin and prevents dehydration, such that the skin remains flexible and is not dried. The hydrophilic salt is absorbed and permeates into the skin.

In some embodiments, since the composition does not contain oily components, the composition improves the penetration of materials such as salts. In addition, since the salts coagulate polymers and organic molecules, it will coagulate and eliminate the oil/fat naturally present on the skin.

In some embodiments, the coagulation of the naturally present fat on the skin supports the water absorption that in turn leads to softening and swelling effect of the skin top layer, the stratum corneum. Without intending to be limited to any particular theory, this will increase the water and salts flux to the skin.

In some embodiments, the solid film creates a hydration effect. For example, by way of illustration, when a person has an oily skin, a prewash with water or soap or cleaning with alcohol prior to applying the coating is beneficial as it increases the permeability of water based cream solutes probably through acting on the stratum corneum.

In some embodiments, the composition also contributes to the debridement of wounds or inflammatory areas assisting the treatment with minerals and with other medications and assisting natural wound healing process.

The following is the theory regarding the physical chemical ion\solute flux mechanism. This is merely a theory and is not meant to limit the present invention. According to Fick's law, the permeation\diffusion (flux of salt into the skin pores and wound) of the mineral salts into the intermediate cell fluids of the skin is predominated by the concentration of the mineral salts. At low concentrations of salts (up to 5%) the governing mechanism is of osmosis, i.e. water molecules of the cells and tissue of the skin will migrate outwards to reduce the exterior salt concentration to equilibrate it to that of the inter cellular fluid and later on to that of the intra cellular fluid. At elevated concentrations of salt, up to 20-30%, the dominant mechanism is still osmosis. Another mechanism, diffusion, emerges but to a lower extent, where the salt ions penetrate the intermediate cell channels and fluid. At high concentrations of salt (at around saturation level), between 30-50%, the dominant mechanism becomes the diffusion and permeation of salt into the skin. The osmosis mechanism is now reduced and suppressed. At extreme concentrations of salt (greater than 50%), the diffusion mechanism is the predominate mechanism; hence, salt permeates into skin, osmosis is negligible. This is amplified at concentrations of greater than70%.

Fick's law determines that increase in concentration increases the flux [μg/(cm2*sec)].

As described above, one example of the present invention facilitates the desired healing flux of the active mineral salts to the skin and inflammatory zone by producing a controlled boundary zone with enabling edge conditions.

Natural sweat, produced by sweat glands, is absorbed by the salt in the solid film, and supports the flux mechanism. In some embodiments, the solid film is configured to absorb sweat from the skin.

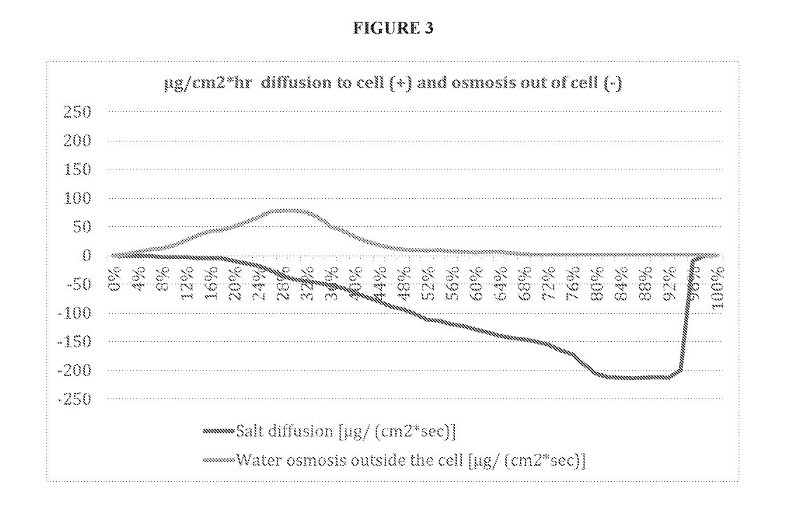

The film serves as an external barrier to exclude exterior contamination yet enabling the skin to continue its natural oxygen and water vapor equilibrium “breathing” process. The boundary conditions, constant inertia of water molecules and the dissolution of salts reservoir, maintain consistent extreme high level concentrations of salts, the driving force for diffusion into the skin (see FIG. 3).

This, on top of the film characteristics described above, provides a theory for the directed ion\solute flux and permeation as well as to the ability of the system to avoid the “dry skin” signs and symptoms associated with many topical medications designed for the treatment of acne and other pathologies, as well as other salt containing formulations.

In colloidal and emulsion science a critical factor for maintaining emulsion system stability is the ionic atmosphere (Debye-Huckel) also known as 1/K (Kappa measured at nanometer and angstroms scale. Typical values for the atmosphericradios, 1/K range at greater than 3 nm, below which the system is unstable and chemical attractions occur between the emulsion micelles that leads to the collapse of the emulsion. The exact expression of this critical radios for chemical and electrical attraction\repulsion is given by the Poisson's equation:

$$1 / K = {[ \frac{{ɛ}_{r} {ɛ}_{0} RT}{2 {F}^{2} {\sum }_{i} {z}_{i}^{2} {C}_{i}} ]}^{1 / 2}$$

Where: 1/K—Atmospheric radios, Kappa
εᵣ—is the relative permittivity of the solvent
εᵣ—the permittivity of free space
R—Gas constant
T—Temperature
F—Faraday constant
zᵢ —ionic valance of ion i
Cᵢ—ionic concentration of ion i
The Poisson equation is used to predict the stability of emulsions in an aqueous solution. In the present invention, atmospheric radius is calculated as follows: ε=80 (aqueous solution) F, R=96 485.3365 s*A/mol and

$$1.3806488 * {10}^{- 23} \frac{{m}^{2} * kg}{{s}^{2} K}$$

respectively (known constants) C [M]—For case study, taken as 50%wt and ion valance of 1 (zᵢ) with average mole weight of 58.45 (NaCl) gives 8.5M.

$$K = {2.32 [ {\sum }_{i} {z}_{i}^{2} {C}_{i} ]}^{1 / 2} = {2.32 [ {\sum }_{1} {1}_{1}^{2} {8.5}_{1} ]}^{1 / 2} = 21.73$$

$$1 / K = 0.046 nm$$

At an atmospheric radius of 0.46 angstrom an emulsion solution renders instable instantly and spontaneously.

### Methods to Treat Skin Conditions According to Some Embodiments of the Present Invention

In some embodiments, the composition achieves the beneficial results of high solute concentration balneotherapy without the need for prolonged immersion of the patient in baths or liquids.

Such skin aesthetic and medical conditions include, but are not limited to, inflammation, infection, wrinkles, wounds, fibroblastic hypertrophy, and other conditions associated with pain, itching, change in skin appearance as in color and contour, and change in skin physical characteristics such as turgor, as well as other signs and symptoms. Skin ailments include, for example, signs and symptoms of autoimmune diseases, psoriasis, atopic dermatitis, aging, acne, vitiligo, scarring, seborrhea, and the like.

In some embodiments, the present invention is a method, comprising:
a. applying the composition to a surface of the skin of a patient suffering from a skin condition, at a site in need of treatment thereof; and
b. allowing the composition to form a solid film at the site where the composition is applied;
   wherein the solid film comprises a concentration of Dead Sea salts at a concentration that is greater than the concentration of the Dead Sea salts in the composition, when the composition was first applied to the skin.

In one embodiment, the Dead Sea salts in the film permeates the skin of the patient.

In one embodiment, the permeation of the Dead Sea salts treats the skin condition.

In one embodiment, the composition is left on the skin for a time sufficient to treat the skin condition.

In one embodiment, the composition is applied in an amount effective to treat the skin condition.

In some embodiments, the patient washes the skin prior to the application of the composition. In some embodiments, the prior washing ensures the skin has absorbed water. The skin is washed with soap.

In some embodiments, the composition is applied once. In some embodiments, the composition is applied a first time, and then a second time. In some embodiments, the second application is carried out after the first application had dried. In some embodiments, the second application is from 10 minutes to 20 minutes after the first application. In some embodiments, the second composition is left to dry for one hour.

In some embodiments, the composition is left on the skin overnight, then removed.

In some embodiments, the composition is applied twice per week. In some embodiments, the composition is applied three times per week. In some embodiments, the composition is applied four times per week.

In some embodiments, the composition is applied at least every hour. In some embodiments, the composition is applied every two hours. In some embodiments, the composition is applied every three hours. In some embodiments, the composition is applied every four hours. In some embodiments, the composition is applied every five hours. In some embodiments, the composition is applied every six hours. In some embodiments, the composition is applied every 12 hours.

In one embodiment, the skin condition is selected from the group consisting of: acne rosacea, psoriasis, rubor, tumor, calor, dolor, scarring, dry skin, aging, wrinkles, inflammation, bacerial infection, and viral infection.

In some embodiments, the composition, when administered to a patient suffering from acne caused at least one effect, selected from the group consisting of:
a. Reduction in wound size and especially decrease in height;
b. Drying of the wound without drying the skin;
c. Change of color of the wounds with reduced red and brightness;
d. Softening of the wounds;
e. Disappearance of the wounds; and
f. Improved hydrated skin appearance.

In some embodiments, the composition, when administered to a patient suffering from psoriasis caused improved appearance of the skin with skin softening.

In some embodiments, the composition, when administered to a patient suffering from facial scarring resulting from chronic acne resulted in improved and fresher/relaxed skin.

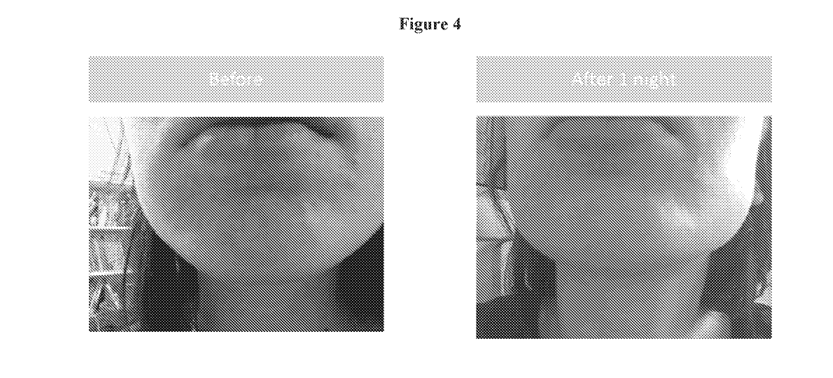

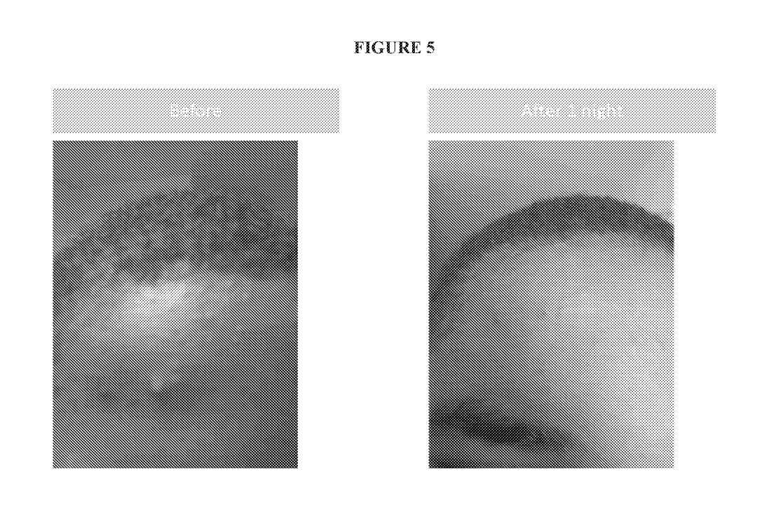

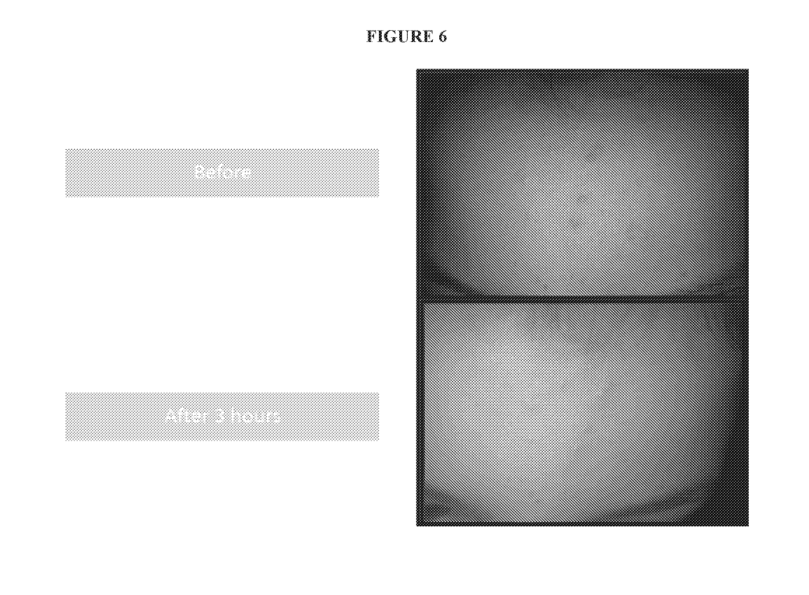

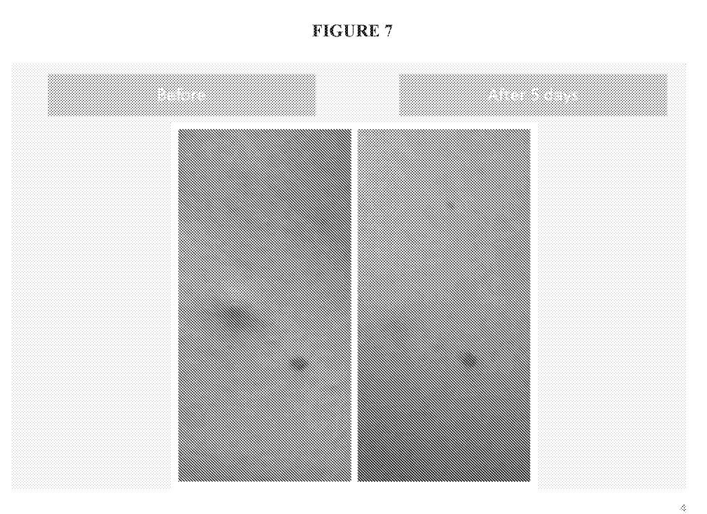

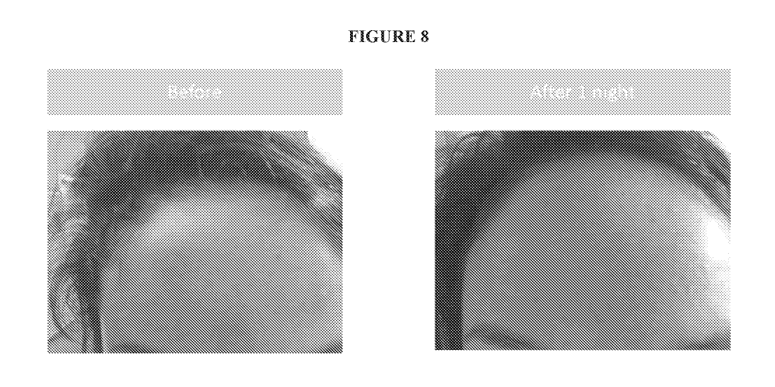

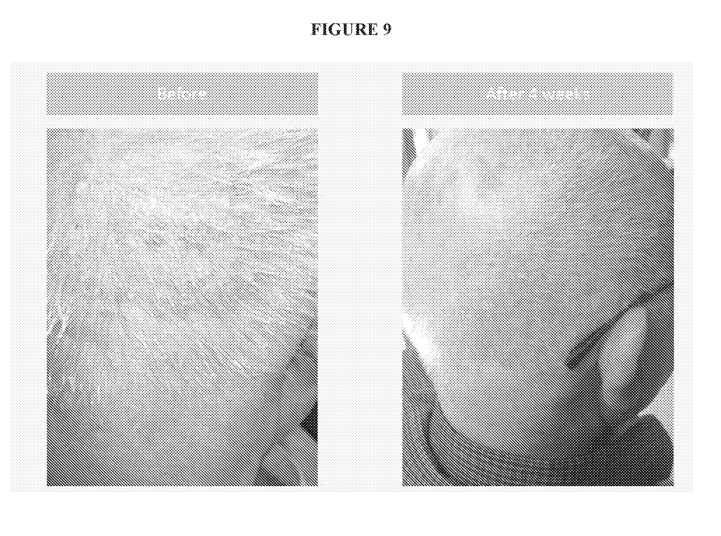

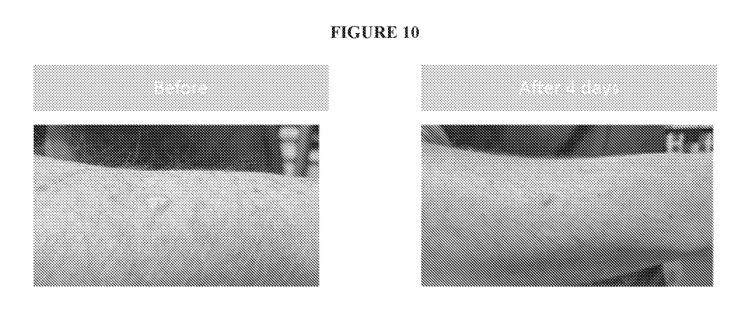

FIGS. 4 through 10 show the effect of treatment according to some embodiments of the present invention.

In one embodiment, the present invention is a method, comprising:
a. applying the composition to a surface of the skin of a patient suffering from comedones, at a site in need of treatment thereof
   wherein the comedones are attached to the skin of the patient;
b. allowing the composition to form a solid film at the site where the composition is applied,
   wherein the solid film comprises a concentration of Dead Sea salts at a concentration that is greater than the concentration of the Dead Sea salts in the composition, when the composition was first applied to the skin,
   wherein the solid film adheres to the skin and to the comedones;
c. leaving the solid film in place for a time sufficient to detach the comedones from the skin of the patient; and
d. removing the solid film, thereby removing the comedones attached to the solid film.

In one embodiment, the time sufficient to detach the comedones from the skin of the patient is from 1 to 3 hours.

In one embodiment, the method is repeated at least once.

“Comedones” or “comedo” as used herein refers to a clogged hair follicle, or sebaceous gland or pore in the skin. Without intending to be limited to any particular theory, sebum and keratin combines with dead skin cells to block the hair follicle or sebaceous gland or pore. When the fat oxidizes the color turns from white to black, hence whiteheads and blackheads.

Without intending to be limited to any particular theory, the composition of the present invention may weaken the adhesion of the comedones with the hair follicle or pore.

Without intending to be limited by any particular theory, the high osmotic pressure in the composition removes the water from the comedones, disrupting the structure of the comedones. The weakened adhesion may then allow the comedones to be mechanically removed, such as, by the solid film, as it is removed from the skin, or by other mechanical means, such as washing, forceps, and the like.

In some embodiments, the method reduced the size of the comedones. In some embodiments, the method changed the color of the blackhead, from black to grey.

In some embodiments, the method reduces the force required to remove the comedones mechanically.

Reference is now made to the following examples, which together with the above descriptions illustrate some embodiments of the invention in a non-limiting fashion.

### EXAMPLES

### Example 1

### Electrical Resistance Measurement as an Indication for Dissolved Salt in the Compositions According to Some Embodiments of the Present Invention

Salts are electrolytes. They dissolve in water to form ions. Conductivity is a measure of how well a solution conducts electricity. To carry a current a solution must contain charged particles, or ions. Most conductivity measurements are made in aqueous solutions, and the ions responsible for the conductivity come from electrolytes dissolved in the water. Salts (like sodium chloride and magnesium sulfate), are all electrolytes. Conductivity is not specific. It measures the total concentration of ions in solution. It cannot distinguish one electrolyte or ion from another.

Conductivity unit is S/m. Conductivity is traditionally determined by measuring the resistance of a solution between two electrodes and is measured by ohm-Ω the electric resistance unit. In the following procedure the resistance measurement serves as the characterization for conductivity.

A MultiMeter Sakal DT-832 is used to determine the resistivity of the composition being tested. During each salt addition step to the composition, the two electrodes are dipped to cover the surface area of the metal detector at a constant gap of 1 cm. The resistivity gage was adjusted to correlate with the scale of resistivity, starting at 2000K Ohm down to 2000 Ohm. The readings were taken after 1 min reaching steady state under no vortex (mixing) at room temperature.

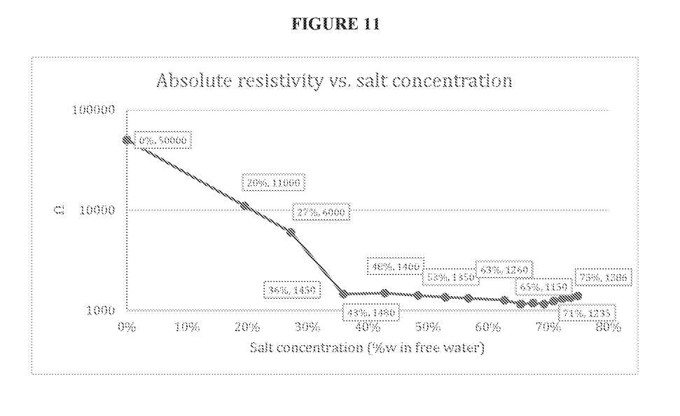

The salt was added gradually to the composition and mixed well. A resistance measurement was taken after each salt addition. The composition was completed after 500 gr salt additions, by adding the last formulation components. Then another resistance measurement was made without any salt addition, this is the composition's absolute resistance. Then additional execs salt was gradually added to the composition at the same manner, up to 75% w salt in the free water of the composition. The measurements for the composition set forth in Table 10 is shown in FIG. 11. The graph depicts the electrical resistance of the dissolved Dead Sea salt, as a function of its salt weight free water solution percent.

From the graph it can be seen that as the salt concentration increases, the absolute resistance decreases, indicating continues rise in the number of dissolved salt ions in the formulation free water. At around 40% w salt solution concentration in composition, the resistance reaches the asymptote of minimum value in the order of 12000Ω, indicating the maximum possible system conductivity. The ions in the solution are at the concentration at which the amount of surrounding water molecule are low and absent, thus the ion are incapable of transferring electrons (the water molecule serve as the cross bridge between the solvated ions). This result is within accordance of the behavior of strong electrolytes ions solutions as described in the literature. In the literature the resistance starts to increase at around 35%-50% salt. This behavior shows that the ions are well dissolved in the solution, significantly beyond what is known in the art, and are kept available for dermal delivery. At this level and onwards the salt, in the form of ions serve as the capacitor of the system, allowing continuous and long lasting reservoir of dissolved ions. The capacitor acts as the “battery” for the accelerated reverse dynamic flow, at which the ions are migrating in to the inner side of the membrane (skin), and as a result reduces the concentration in the outer layer.

### Example 2

### Electrical Resistance Measurement as an Indication for Dissolved Salt in the Compositions According to Some Embodiments of the Present Invention

A MultiMeter Sakal DT-832 is used to determine the resistivity of the hand skin before and after application of the composition set forth in Table 10, as follows:
The skin hand was washed and cleaned with tap water and left slightly wet.
The two electrodes were tightly placed on the wet skin in 1 cm distance. The resistivity gage was adjusted to correlate with the scale of resistivity at 2000K Ohm. The readings were taken after 1 min reaching steady state.
Wet skin resistance: 630,000 Ω
One gr of the composition described in Table 10 was applied on 2.5 cm×2.5 cm surface of clean dry hand skin. After three hours the formulation was completely removed and washed away with tap water. The skin was left slightly wet.
The two electrodes were tightly placed on the wet skin in 1 cm distance. The resistivity gage was adjusted to correlate with the scale of resistivity at 2000K Ohm. The readings were taken after 1 min reaching steady state.
Wet skin resistance after ilumi preparation formulation application was: 65,000 Ω.

The resistance decreases, of the order of magnitude, measured on the skin, after the application and washing the formulation, can be explained only by the penetration and the presence of salt dissolved ions with absorbed water in the top skin layer. This measurement demonstrates in practice that the formulation on the skin serves as the ion capacitator system. This “capacitor” promotes the ion dissolved flow and penetration in to the skin as explained above.

### Example 3

### Treatment of Acne Vulgaris According to Some Embodiments of the Present Invention

A 16 year old male presented with acne vulgaris. The patient was treated with the compositon described in Table 2 overnight. Prior to treatment, the patient first cleansed the skin with soap and water. The patient exhibited dramatic improvement that continued and became even more pronounce after the 3rd and 4th treatments.

### Example 4

### Treatment of Mild Acne According to Some Embodiments of the Present Invention

A 40 year old female presented with mild acne during menstruation, consisting of 2-4 lesions, often associated with a crater. The patient was treated with the composition described in Table 2 overnight. Prior to treatment, the patient first cleansed the skin with soap and water. The patient exhibited dramatic therapeutic effects after 1-2 nights of treatment.

### Example 5

### Treatment of Psoriasis According to Some Embodiments of the Present Invention

A 70 year old female presented with mild psoriasis with only dermal expression usually in the antecubital areas. The patient was treated with the composition described in Table 3, coated on a sleeve. The formula was applied on the internal part of the sleeve by spraying with an industrial machine. The sleeve was cut from a tight sports shirt with a flexible breathing fabric. The patient was instructed to wear the sleeve through-out the night. In the morning, the patient reported that her skin was less red and much softer. The patient was highly pleased and her main complaint was that she has not received a sleeve for her other arm and that she had not received more sleeves. The effect lasted for two days. An interesting observation was that at the beginning the color of the coating was white but after use it was almost transparent. It was also lighter by weight.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

What is claimed is:

1. A composition comprising:
a. a water-based polymer emulsion from 10 wt % to 90 wt % of the composition; and
b. Dead Sea salt from 5 wt % to 80 wt % of the composition.

2. The composition of claim 1, further comprising a wetting and dispersing additive up to 20 wt % of the composition.

3. The composition of claim 2, wherein the wetting and dispersing additive is the wetting and dispersing additive sold under the tradename DISPERBYK®.

4. The composition of claim 1, further comprising a smectite clay up to 30 wt % of the composition.

5. The composition of claim 5, wherein the smectite clay is the smectite clay sold under the tradename BENTONE® EW.

6. The composition of claim 1, further comprising a rheology modifier up to 30 wt % of the composition.

7. The composition of claim 6, wherein the rheology modifier is the rheology modifier sold under the tradename TEGO® Rheo8600.

8. The composition of claim 1, further comprising a defoamer up to 8 wt % of the composition.

9. The composition of claim 8, wherein the defoamer is the defoamer sold under the tradename TEGO® Foamex825.

10. The composition of claim 1, further comprising water up to 90 wt % of the composition.

11. The composition of claim 1, further comprising at least one additional component selected from the group consisting of: a humectant, an alcohol, an adhesion promoter, a lubricant, a softening agent, a fragrance, and a therapeutic agent.

12. The composition of claim 11, wherein the pigment is up to 20 wt % of the composition.

13. The composition of claim 11, wherein the fragrance is up to 5 wt % of the composition.

14. The composition of claim 11, wherein the therapeutic agent is an anti-bacterial agent.

15. The composition of claim 14, wherein the antibacterial agent is selected from the group consisting of salicylic acid and titanium dioxide.

16. The composition of claim 15, wherein the antibacterial agent is up to 15 wt % of the composition.

17. A method, comprising:
a. applying the composition of claim 1 to a surface of the skin of a patient suffering from a skin condition, at a site in need of treatment thereof; and
b. allowing the composition to form a solid film at the site where the composition is applied;
wherein the solid film comprises a concentration of Dead Sea salts at a concentration that is greater than the concentration of the Dead Sea salts in the composition, when the composition was first applied to the skin.

18. The method of claim 18, wherein the Dead Sea salts in the film permeates the skin of the patient.

19. The method of claim 18, wherein the permeation of the Dead Sea salts treats the skin condition.

20. The method of claim 18, wherein the composition is left on the skin for a time sufficient to treat the skin condition.

21. The method of claim 18, wherein the composition is applied in an amount effective to treat the skin condition.

22. The method of claim 18, where in the skin condition is selected from the group consisting of: acne rosacea, acne vulgaris, psoriasis, rubor, tumor, calor, dolor, scarring, dry skin, aging, wrinkles, inflammation, bacerial infection, and viral infection.

23. A method comprising:
a. applying the composition to a surface of the skin of a patient suffering from comedones, at a site in need of treatment thereof,
wherein the comedones are attached to the skin of the patient;
b. allowing the composition to form a solid film at the site where the composition is applied,
wherein the solid film comprises a concentration of Dead Sea salts at a concentration that is greater than the concentration of the Dead Sea salts in the composition, when the composition was first applied to the skin,
wherein the solid film adheres to the skin and to the comedones;
c. leaving the solid film in place for a time sufficient to detach the comedones from the skin of the patient; and
d. removing the solid film, thereby removing the comedones attached to the solid film.

24. The method of claim 23, wherein the time sufficient to detach the comedones from the skin of the patient is from 1 to 3 hours.

25. The method of claim 23, wherein the method is repeated at least once.
